(19) United States
(12) Patent Application Publication
(10) Pub. No.: US 2006/0153875 A1
(43) Pub. Date: Jul. 13, 2006

(54) Novel brachyspira hyodyseteriae vaccine

(76) Inventors: Ben Adler, Vic (AU); Paul Antony Cullen, Vic (AU); Scott Adam James Coutts, Vic (AU); Dieter Mark Bulach, Vic (AU); Ruud Phillip Antoon Maria Segers, Boxmeer (NL)

Correspondence Address: INTERVET INC.;PATENT DEPARTMENT, PO BOX 318, MILLSBORO, DE, 19966-0318, US

(21) Appl. No.: 10/521,693

(22) PCT Filed: Jul. 11, 2003
(86) PCT No.: PCT/EP03/07611

§ 371 Date: Sep. 12, 2005

(30) Foreign Application Priority Data
Jul. 17, 2002 (EP) 02077882.5

(51) Int. Cl. A61K 39/02; C07H 21/04; C12P 21/06; C07K 14/195; C12N 15/74

(52) U.S. Cl. 424/234.1; 536/23.7; 530/359; 435/69.1; 435/252.3; 435/471

(57) ABSTRACT

The present invention relates to nucleic acid sequences encoding a 61 kD and a 20 kD Brachyspira hyodysenteriae lipoprotein and to parts of such nucleic acid sequences that encode an immunogenic fragment of such lipoproteins, and to DNA fragments, recombinant DNA molecules, live recombinant carriers and host cells comprising such nucleic acid sequences or such parts thereof. The invention also relates to a 61 kD and a 20 kD Brachyspira hyodysenteriae lipoprotein and immunogenic parts thereof encoded by such sequences. Furthermore, the present invention relates to vaccines comprising such nucleic acid sequences and parts thereof, DNA fragments, recombinant DNA molecules, live recombinant carriers and host cells comprising such nucleic acid sequences or such parts thereof, lipoproteins or immunogenic parts thereof and antibodies against such lipoproteins or immunogenic parts thereof. Also, the invention relates to the use of said lipoproteins in vaccines and for the manufacture of vaccines. Moreover, the invention relates to the use of said nucleic acid sequences, lipoproteins or antibodies for diagnostic or vaccination purposes. Finally the invention relates to diagnostic kits comprising such a nucleic acid, lipoprotein or antibodies against such lipoprotein.

The present invention relates to nucleic acid sequences encoding a Brachyspira hyodysenteriae lipoprotein and to parts of such nucleic acid sequences that encode an immunogenic fragment of such lipoproteins, and to DNA fragments, recombinant DNA molecules, live recombinant carriers and host cells comprising such nucleic acid sequences or such parts thereof. The invention also relates to a Brachyspira hyodysenteriae lipoprotein and immunogenic parts thereof encoded by such sequences. Furthermore, the present invention relates to vaccines comprising such nucleic acid sequences and parts thereof, DNA fragments, recombinant DNA molecules, live recombinant carriers and host cells comprising such nucleic acid sequences or such parts thereof, lipoproteins or immunogenic parts thereof and antibodies against such lipoproteins or immunogenic parts thereof. Also, the invention relates to the use of said lipoproteins in vaccines and for the manufacture of vaccines. Moreover, the invention relates to the use of said nucleic acid sequences, lipoproteins or antibodies for diagnostic or vaccination purposes. Finally the invention relates to diagnostic kits comprising such a nucleic acid, lipoprotein or antibodies against such lipoprotein.

Brachyspira hyodysenteriae is an anaerobic, oxygen tolerant, Gram-negative spirochete that is strongly β-hemolytic. In the past, Brachyspira hyodysenteriae was also known as Treponema hyodysenteriae and Serpulina hyodysenteriae. It is the etiological agent of swine dysentery, a mucohemorrhagic diarrheal disease of post-weaning pigs. Infection in swine with this bacterium can be suppressed with antimicrobials. However, recent restrictions on the use of antibiotics in animal feed provide impetus for the identification of candidate vaccine antigens as alternatives to the use of antimicrobials.

Swine dysentery (SD) is a mucohemorrhagic diarrheal disease of post-weaning pigs. SD has a major economic impact worldwide. The severity of the symptoms is variable between individuals and herds. The first signs of infection include soft, yellow to gray faeces, loss of appetite and increased rectal temperature in some animals. Subsequent to this, the faeces begin to contain flecks of blood and plugs of mucus. As the disease progresses, the faeces become watery, and prolonged diarrhea may lead to death by dehydration. Faeces containing B. hyodysenteriae are ingested by susceptible pigs, after which the organisms survive passage through the acidic conditions of the stomach and reach the large intestine. Experimental evidence suggests that the organism has a chemotactic response to mucus, enabling it to navigate to the colon mucosa where it invades the colon crypts. The large intestine is the major site for water and electrolyte resorption in pigs; damage to the large intestine thus results in colon absorption failure and dehydration.

Diagnosis of SD is based on clinical signs, herd history and isolation of B. hyodysenteriae on selective medium. B. hyodysenteriae is often difficult to isolate because of its slow growth and anaerobic requirements, a problem exacerbated by poor storage and transportation of samples. Even when isolation is possible, biochemical tests of isolates are unable to differentiate between B. hyodysenteriae and B. innocens, a non-pathogenic intestinal spirochete. The costly and time consuming nature of enter pathogenic studies in pigs or suitable animal models (such as mice, guinea pigs and chickens) precludes this approach for regular diagnosis.

Several virulence factors of B. hyodysenteriae have been identified and their role in the pathogenesis of swine dysentery investigated. For example, the initial colonization of the colon by B. hyodysenteriae is facilitated by its chemotactic response to mucus. (Kennedy, M. J., D. K. Rosnick, R. G Ulrich, and R. J. Yancey. 1988, J. Gen. Microbiol. 134:1565-1576). (Kennedy, M. J., and R. J. Yancey. 1996, Vet. Microbiol. 49:21-30).

The importance of chemotaxis was demonstrated by Rosey, (Rosey, E. L., M. J. Kennedy, and R. J. Yancey, Jr. 1996, Infect. Immun. 64:4154-4162), who showed that a dual flagella mutant was severely attenuated in a murine model. Once the colonization of the swine caecum is established, NADH oxidase is thought to protect the Brachyspira from oxygen toxicity. (Stanton, T. B., and N. S. Jensen. 1993, J. Bacteriol. 175:2980-2987). (Stanton, T. B., and R. Sellwood. 1999, Anaerobe 5:539-546). This hypothesis is supported by the observation that an NADH oxidase mutant exhibited reduced colonization of the swine caecum. The caecal lesions apparent on pathological examination of chronically infected swine can be induced by administration of B. hyodysenteriae haemolysin-containing extracts. Initially, three distinct putative haemolysin genes, tlyA, tlyB and tlyC were cloned and sequenced. (Muir, S., M. B. Koopman, S. J. Libby, L. A. Joens, F. Heffron, and J. G. Kusters. 1992, Infect. Immun. 60:529-535). (ter Huurne, A. A., S. Muir, M. van Houten, B. A. van der Zeijst, W. Gaastra, and J. G. Kusters. 1994, Microb. Pathog. 16:269-282). A recent report by Hsu et al. has cast doubt on whether the tly genes actually encode haemolysins and has implicated another gene hlyA in haemolysin production (Hsu, T., D. L. Hutto, F. C. Minion, R. L. Zuerner, and M. J. Wannemuehler. 2001, Infect. Immun. 69:706-711).

In the search for antigens which elicit a protective immune response, several proteins have been identified which localize to the outer membrane of B. hyodysenteriae. A Proteinase K sensitive 16-kDa antigen was localized to the outer membrane. Subsequently the gene encoding this antigen, smpA, was cloned and found not to be expressed in vivo (Thomas, W., R. Sellwood, and R. J. Lysons. 1992, Infect. Immun. 60:3111-3116). (Sellwood, R., F. Walton, W. Thomas, M. R. Burrows, and J. Chesham. 1995, Vet. Microbiol. 44:25-35). An extracytoplasmic 39-kDa antigen, Vsp39, was identified by surface iodination as the predominant surface component of B. hyodysenteriae (Gabe, J. D., R. E. Chang, R. J. Slomiany, W. H. Andrews, and M. T. Mccaman. 1995, Infect. Immun. 63:142-148). While the gene encoding Vsp39 has not been cloned, a series of related tandem paralogous genes encoding 39-kDa proteins with 83-90% identity was identified (Gabe, J. D., E. Dragon, R. J. Chang, and M. T. McCaman. 1998, Identification of a linked set of genes in Serpulina hyodysenteriae (B204) predicted to encode closely related 39-kilodalton extracytoplasmic proteins. J. Bacteriol. 180:444-448). (McCaman, M. T., K. Auer, W. Foley, and J. D. Gabe. 1999, Vet. Microbiol. 68:273-283). A putative 30-kDa lipoprotein, BmpB, was found to react with convalescent pig sera. No further data of this protein have been published however (Lee, B. J., T. La, A. S. Mikosza, and D. J. Hampson. 2000, Vet. Microbiol. 76:245-257).

It is therefore clear that there is a need for new and effective vaccines, especially vaccines that provide broad protection.

It is an objective of the present invention to provide novel vaccines for combating Brachyspira hyodysenteriae infections.

Two novel genes have now surprisingly been found, which are thought to encode novel surface expressed bacterial lipoproteins. These lipoproteins, BlpB and BlpC turn out to be suitable vaccine components, alone and especially in combination with each other, in vaccines for combating Brachyspira hyodysenteriae infections. Both genes have now been cloned and sequenced and their sequences are depicted in SEQ ID NO: 1 (BlpB) and SEQ ID NO: 3 (BlpC). The first ORF, blpB encodes a lipoprotein of 537 amino acids with a molecular mass of 61 kD (as depicted in SEQ ID NO: 2). The second ORF, blpC encodes a lipoprotein of 179 amino acids with a molecular mass of 20 kD (as depicted in SEQ ID NO: 4).

It is well-known in the art, that many different nucleic acid sequences can encode one and the same protein. This phenomenon is commonly known as wobble in the second and especially the third base of each triplet encoding an amino acid. This phenomenon can result in a heterology of about 30% for two nucleic acid sequences still encoding the same protein. Therefore, two nucleic acid sequences having a sequence homology of about 70% can still encode one and the same protein.

Thus, one embodiment relates to a nucleic acid sequence encoding a 61 kD Brachyspira hyodysenteriae lipoprotein or a part of said nucleic acid sequence that encodes an immunogenic fragment of said lipoprotein wherein said nucleic acid sequence or said part thereof has at least 70% homology with the nucleic acid sequence of the Brachyspira hyodysenteriae lipoprotein gene as depicted in SEQ ID NO: 1.

The 61 molecular weight is determined in gel electrophoresis sodium dodecyl sulphate on a polyacryl amide gel (SDS-PAGE). Due to slight variability of molecular weight determination frequently encountered in the art, the molecular weight can vary between 56 an 66 kD. Therefore the molecular weight of the lipoproteins according to the invention should be interpreted as to be 61+/−5 kD.

Preferably, a nucleic acid sequence according to the invention encoding this 61 Brachyspira hyodysenteriae lipoprotein or a part of that nucleic acid sequence that encodes an immunogenic fragment of that lipoprotein has at least 80%, preferably 90%, more preferably 95% homology with the nucleic acid sequence of the Brachyspira hyodysenteriae lipoprotein gene as depicted in SEQ ID NO: 1.

Even more preferred is a homology level of 98%, 99% or even 100%.

The level of nucleotide homology can be determined with the computer program “BLAST 2 SEQUENCES” by selecting sub-program: “BLASTN” that can be found at www.ncbi.nlm.nih.gov/blast/bl2seq/bl2.html.

A reference for this program is Tatiana A. Tatusova, Thomas L. Madden FEMS Microbiol. Letters 174: 247-250 (1999). Parameters used are the default parameters:

Reward for a match: +1. Penalty for a mismatch: −2. Open gap: 5. Extension gap: 2. Gap x_dropoff: 50.

Another embodiment relates to a nucleic acid sequence encoding a 20 kD Brachyspira hyodysenteriae lipoprotein or a part of said nucleic acid sequence that encodes an immunogenic fragment of said lipoprotein wherein said nucleic acid sequence or said part thereof has at least 70% homology with the nucleic acid sequence of the Brachyspira hyodysenteriae lipoprotein gene as depicted in SEQ ID NO: 3.

The 20 kD molecular weight is determined in gel electrophoresis on a polyacryl amide gel. Due to slight variability of molecular weight determination frequently encountered in the art, the molecular weight can vary between 15 and 25 kD. Therefore the molecular weight of the lipoproteins according to the invention should be interpreted as to be 20+/−5 kD.

Preferably, a nucleic acid sequence according to the invention encoding this 20 kD Brachyspira hyodysenteriae lipoprotein or a part of that nucleic acid sequence that encodes an immunogenic fragment of that lipoprotein has at least 80%, preferably 90%, more preferably 95% homology with the nucleic acid sequence of the Brachyspira hyodysenteriae lipoprotein gene as depicted in SEQ ID NO: 3.

Even more preferred is a homology level of 98%, 99% or even 100%. Nucleotide sequences that are complementary to the sequence depicted in SEQ ID NO 1 or SEQ ID NO 3 or nucleotide sequences that comprise tandem arrays of the sequences according to the invention are also within the scope of the invention.

Since the present invention discloses nucleic acid sequences encoding novel 61 kD and 20 kD Brachyspira hyodysenteriae lipoproteins, it is now for the first time possible to obtain these proteins in sufficient quantities. This can e.g. be done by using expression systems to express the whole or parts of a gene encoding the protein or an immunogenic fragment thereof.

Therefore, in a more preferred form of this embodiment, the invention relates to DNA fragments comprising a nucleic acid sequence according to the invention. A DNA fragment is a stretch of nucleotides that functions as a carrier for a nucleic acid sequence according to the invention. Such DNA fragments can e.g. be plasmids, into which a nucleic acid sequence according to the invention is cloned. Such DNA fragments are e.g. useful for enhancing the amount of DNA for use as a primer and for expression of a nucleic acid sequence according to the invention, as described below.

An essential requirement for the expression of the nucleic acid sequence is an adequate promoter functionally linked to the nucleic acid sequence, so that the nucleic acid sequence is under the control of the promoter. It is obvious to those skilled in the art that the choice of a promoter extends to any eukaryotic, prokaryotic or viral promoter capable of directing gene transcription in cells used as host cells for protein expression.

Therefore, an even more preferred form of this embodiment relates to a recombinant DNA molecule comprising a DNA fragment and/or a nucleic acid sequence according to the invention wherein the nucleic acid sequence according to the invention is placed under the control of a functionally linked promoter. This can be obtained by means of e.g. standard molecular biology techniques. (Maniatis/Sambrook (Sambrook, J. Molecular cloning: a laboratory manual, 1989. ISBN 0-87969-309-6).

Functionally linked promoters are promoters that are capable of controlling the transcription of the nucleic acid sequences to which they are linked.

Such a promoter can be the native promoter of the novel gene or another promoter of Brachyspira, provided that that promoter is functional in the cell used for expression. It can also be a heterologous promoter. When the host cells are bacteria, useful expression control sequences which may be used include the Trp promoter and operator (Goeddel, et al., Nucl. Acids Res., 8, 4057,1980); the lac promoter and operator (Chang, et al., Nature, 275, 615, 1978); the outer membrane protein promoter (Nakamura, K. and Inouge, M., EMBO J., 1, 771-775, 1982); the bacteriophage lambda promoters and operators (Remaut, E. et al., Nucl. Acids Res., 11, 4677-4688, 1983); the α-amylase (B. subtilis) promoter and operator, termination sequences and other expression enhancement and control sequences compatible with the selected host cell.

When the host cell is yeast, useful expression control sequences include, e.g., α-mating factor. For insect cells the polyhedrin or p10 promoters of baculoviruses can be used (Smith, G. E. et al., Mol. Cell. Biol. 3, 2156-65, 1983). When the host cell is of vertebrate origin illustrative useful expression control sequences include the (human) cytomegalovirus immediate early promoter (Seed, B. et al., Nature 329, 840-842,1987; Fynan, E. F. et al., PNAS 90, 11478-11482,1993; Ulmer, J. B. et al., Science 259, 1745-1748, 1993), Rous sarcoma virus LTR (RSV, Gorman, C. M. et al., PNAS 79, 6777-6781, 1982; Fynan et al., supra; Ulmer et al., supra), the MPSV LTR (Stacey et al., J. Virology 50, 725-732, 1984), SV40 immediate early promoter (Sprague J. et al., J. Virology 45, 773, 1983), the SV-40 promoter (Berman, P. W. et al., Science, 222, 524-527, 1983), the metallothionein promoter (Brinster, R. L. et al., Nature 296, 39-42, 1982), the heat shock promoter (Voellmy et al., Proc. Natl. Acad. Sci. USA, 82, 4949-53, 1985), the major late promoter of Ad2 and the β-actin promoter (Tang et al., Nature 356, 152-154, 1992). The regulatory sequences may also include terminator and poly-adenylation sequences. Amongst the sequences that can be used are the well known bovine growth hormone poly-adenylation sequence, the SV40 poly-adenylation sequence, the human cytomegalovirus (hCMV) terminator and poly-adenylation sequences.

Bacterial, yeast, fungal, insect and vertebrate cell expression systems are very frequently used systems. Such systems are well-known in the art and generally available, e.g. commercially through Clontech Laboratories, Inc. 4030 Fabian Way, Palo Alto, Calif. 94303-4607, USA. Next to these expression systems, parasite-based expression systems are attractive expression systems. Such systems are e.g. described in the French Patent Application with Publication number 2 714 074, and in U.S. NTIS Publication No U.S. Ser. No. 08/043,109 (Hoffman, S. and Rogers, W.: Public. Date 1 Dec. 1993).

A still even more preferred form of this embodiment of the invention relates to Live Recombinant Carriers (LRCs) comprising a nucleic acid sequence encoding a 61 kD and/or 20 kD Brachyspira hyodysenteriae lipoprotein or an immunogenic fragment thereof according to the invention, a DNA fragment according to the invention or a recombinant DNA molecule according to the invention. These LRCs are micro-organisms or viruses in which additional genetic information, in this case a nucleic acid sequence encoding the 61 kD and/or 20 kD Brachyspira hyodysenteriae lipoprotein or an immunogenic fragment thereof according to the invention has been cloned. Pigs infected with such LRCs will produce an immunological response not only against the immunogens of the carrier, but also against the immunogenic parts of the protein(s) for which the genetic code is additionally cloned into the LRC, e.g. the novel 61 kD and/or 20 kD Brachyspira hyodysenteriae lipoprotein gene according to the invention.

As an example of bacterial LRCs, attenuated Salmonella strains known in the art can very attractively be used.

Also, live recombinant carrier parasites have i.a. been described by Vermeulen, A. N. (Int. Journ. Parasitol. 28: 1121-1130 (1998)).

Furthermore, LRC viruses may be used as a way of transporting the nucleic acid sequence into a target cell. Live recombinant carrier viruses are also called vector viruses. Viruses often used as vectors are Vaccinia viruses (Panicali et al; Proc. Natl. Acad. Sci. USA, 79: 4927 (1982), Herpesviruses (E.P.A. 0473210A2), and Retroviruses (Valerio, D. et al; in Baum, S. J., Dicke, K. A., Lotzova, E. and Pluznik, D. H. (Eds.), Experimental Haematology today—1988. Springer Verlag, New York: pp. 92-99 (1989)).

The technique of in vivo homologous recombination, well-known in the art, can be used to introduce a recombinant nucleic acid sequence into the genome of a bacterium, parasite or virus of choice, capable of inducing expression of the inserted nucleic acid sequence according to the invention in the host animal.

Finally another form of this embodiment of the invention relates to a host cell comprising a nucleic acid sequence encoding a protein according to the invention, a DNA fragment comprising such a nucleic acid sequence or a recombinant DNA molecule comprising such a nucleic acid sequence under the control of a functionally linked promoter. This form also relates to a host cell containing a live recombinant carrier comprising a nucleic acid molecule encoding a 61 kD and/or 20 kD Brachyspira hyodysenteriae lipoprotein or an immunogenic fragment thereof according to the invention.

A host cell may be a cell of bacterial origin, e.g. Escherichia coli, Bacillus subtilis and Lactobacillus species, in combination with bacteria-based plasmids as pBR322, or bacterial expression vectors as pGEX, or with bacteriophages. The host cell may also be of eukaryotic origin, e.g. yeast-cells in combination with yeast-specific vector molecules, or higher eukaryotic cells like insect cells (Luckow et al; Bio-technology 6: 47-55 (1988)) in combination with vectors or recombinant baculoviruses, plant cells in combination with e.g. Ti-plasmid based vectors or plant viral vectors (Barton, K. A. et al; Cell 32: 1033 (1983), mammalian cells like Hela cells, Chinese Hamster Ovary cells (CHO) or Crandell Feline Kidney-cells, also with appropriate vectors or recombinant viruses.

Another embodiment of the invention relates to the novel 61 kD and/or 20 kD Brachyspira hyodysenteriae lipoprotein and to immunogenic fragments thereof according to the invention.

The concept of immunogenic fragments will be defined below.

One form of this embodiment relates to a 61 kD Brachyspira hyodysenteriae lipoprotein and to immunogenic fragments thereof, having an amino acid sequence homology of at least 70% with the amino acid sequence as depicted in SEQ ID NO: 2.

In a preferred form, the embodiment relates to such Brachyspira lipoproteins and immunogenic fragments thereof, that have a sequence homology of at least 80%, preferably 90%, more preferably 95% homology to the amino acid sequence as depicted in SEQ ID NO: 2.

Even more preferred is a homology level of 98%, 99% or even 100%.

Another form of this embodiment relates to a 20 kD Brachyspira hyodysenteriae lipoprotein and to immunogenic fragments thereof, having an amino acid sequence homology of at least 70% with the amino acid sequence as depicted in SEQ ID NO: 4.

In a preferred form, the embodiment relates to such Brachyspira lipoproteins and immunogenic fragments thereof, that have a sequence homology of at least 80%, preferably 90%, more preferably 95% homology to the amino acid sequence as depicted in SEQ ID NO: 4.

Even more preferred is a homology level of 98%, 99% or even 100%.

Another form of this embodiment relates to such 61 kD and/or 20 kD Brachyspira hyodysenteriae lipoproteins and immunogenic fragments of said protein encoded by a nucleic acid sequence according to the invention.

The level of protein homology can be determined with the computer program “BLAST 2 SEQUENCES” by selecting sub-program: “BLASTP”, that can be found at www.ncbi.nlm.nih.gov/blast/bl2seq/bl2.html.

A reference for this program is Tatiana A. Tatusova, Thomas L. Madden FEMS Microbiol. Letters 174: 247-250 (1999). Matrix used: “blosum62”. Parameters used are the default parameters:

Open gap: 11. Extension gap: 1. Gap x_dropoff: 50.

It will be understood that, for the particular proteins embraced herein, natural variations can exist between individual Brachyspira strains. These variations may be demonstrated by (an) amino acid difference(s) in the overall sequence or by deletions, substitutions, insertions, inversions or additions of (an) amino acid(s) in said sequence. Amino acid substitutions which do not essentially alter biological and immunological activities, have been described, e.g. by Neurath et al in “The Proteins” Academic Press New York (1979). Amino acid replacements between related amino acids or replacements which have occurred frequently in evolution are, inter alia, Ser/Ala, Ser/Gly, Asp/Gly, Asp/Asn, Ile/Val (see Dayhof, M. D., Atlas of protein sequence and structure, Nat. Biomed. Res. Found., Washington D.C., 1978, vol. 5, suppl. 3). Other amino acid substitutions include Asp/Glu, Thr/Ser, Ala/Gly, Ala/Thr, Ser/Asn, Ala/Val, Thr/Phe, Ala/Pro, Lys/Arg, Leu/Ile, Leu/Val and Ala/Glu. Based on this information, Lipman and Pearson developed a method for rapid and sensitive protein comparison (Science, 227, 1435-1441, 1985) and determining the functional similarity between homologous proteins. Such amino acid substitutions of the exemplary embodiments of this invention, as well as variations having deletions and/or insertions are within the scope of the invention as long as the resulting proteins retain their immune reactivity. This explains why Brachyspira lipoproteins according to the invention, when isolated from different field isolates, may have homology levels of about 70%, while still representing the same protein with the same immunological characteristics.

Those variations in the amino acid sequence of a certain protein according to the invention that still provide a protein capable of inducing an immune response against infection with Brachyspira hyodysenteriae or at least against the clinical manifestations of the infection are considered as “not essentially influencing the immunogenicity”.

When a protein is used for e.g. vaccination purposes or for raising antibodies, it is however not necessary to use the whole protein. It is also possible to use a fragment of that protein that is capable, as such or coupled to a carrier such as e.g. KLH, of inducing an immune response against that protein, a so-called immunogenic fragment. An “immunogenic fragment” is understood to be a fragment of the full-length protein that still has retained its capability to induce an immune response in a vertebrate host, e.g. comprises a B- or T-cell epitope. Shortly, an immunogenic fragment is a fragment that is capable of inducing an antigenic response against the 61 or the 20 Brachyspira hyodysenteriae lipoprotein according to the invention. At this moment, a variety of techniques is available to easily identify DNA fragments encoding antigenic fragments (determinants). The method described by Geysen et al (Patent Application WO 84/03564, Patent Application WO 86/06487, U.S. Pat. No. 4,833,092, Proc. Natl. Acad. Sci. 81: 3998-4002 (1984), J. Imm. Meth. 102, 259-274 (1987), the so-called PEPSCAN method is an easy to perform, quick and well-established method for the detection of epitopes; the immunologically important regions of the protein. The method is used world-wide and as such well-known to man skilled in the art. This (empirical) method is especially suitable for the detection of B-cell epitopes. Also, given the sequence of the gene encoding any protein, computer algorithms are able to designate specific protein fragments as the immunologically important epitopes on the basis of their sequential and/or structural agreement with epitopes that are now known. The determination of these regions is based on a combination of the hydrophilicity criteria according to Hopp and Woods (Proc. Natl. Acad. Sci. 78: 38248-3828 (1981)), and the secondary structure aspects according to Chou and Fasman (Advances in Enzymology 47: 45-148 (1987) and U.S. Pat. No. 4,554,101). T-cell epitopes can likewise be predicted from the sequence by computer with the aid of Berzofsky's amphiphilicity criterion (Science 235, 1059-1062 (1987) and U.S. Patent application NTIS US 07/005,885). A condensed overview is found in: Shan Lu on common principles: Tibtech 9: 238-242 (1991), Good et al on Malaria epitopes; Science 235: 1059-1062 (1987), Lu for a review; Vaccine 10: 3-7 (1992), Berzofsky for HIV-epitopes; The FASEB Journal 5:2412-2418 (1991). An immunogenic fragment usually has a minimal length of 8 amino acids, preferably more then 8, such as 9, 10, 12, 15 or even 20 amino acids. The nucleic acid sequences encoding such a fragment therefore have a length of at least 24, but preferably 27, 30, 36, 45 or even 60 nucleic acids.

Therefore, one form of still another embodiment of the invention relates to vaccines for combating Brachyspira hyodysenteriae infection, that comprise a 61 kD and/or 20 kD Brachyspira hyodysenteriae protein or immunogenic fragments thereof, according to the invention as described above together with a pharmaceutically acceptable carrier.

Still another embodiment of the present invention relates to the 61 kD and/or 20 kD Brachyspira hyodysenteriae protein according to the invention or immunogenic fragments thereof for use in a vaccine.

Still another embodiment of the present invention relates to the use of a nucleic acid sequence, a DNA fragment, a recombinant DNA molecule, a live recombinant carrier, a host cell or a lipoprotein or an immunogenic fragment thereof according to the invention for the manufacturing of a vaccine for combating Brachyspira hyodysenteriae infection.

One way of making a vaccine according to the invention is by growing the bacteria, followed by biochemical purification of the 61 kD and/or 20 kD Brachyspira hyodysenteriae lipoprotein or immunogenic fragments thereof, from the bacterium. This is however a very time-consuming way of making the vaccine.

It is therefore much more convenient to use the expression products of the gene encoding a 61 kD and/or 20 kD Brachyspira hyodysenteriae lipoprotein or immunogenic fragments thereof in vaccines. This is possible for the first time now because the nucleic acid sequence of the genes encoding a 61 kD and 20 kD lipoprotein are provided in the present invention.

Vaccines based upon the expression products of these genes can easily be made by admixing the protein according to the invention or immunogenic fragments thereof according to the invention with a pharmaceutically acceptable carrier as described below.

Alternatively, a vaccine according to the invention can comprise live recombinant carriers as described above, capable of expressing the protein according to the invention or immunogenic fragments thereof. Such vaccines, e.g. based upon a Salmonella carrier or a viral carrier e.g. a Herpesvirus vector have the advantage over subunit vaccines that they better mimic the natural way of infection of Brachyspira hyodysenteriae. Moreover, their self-propagation is an advantage since only low amounts of the recombinant carrier are necessary for immunization.

Vaccines can also be based upon host cells as described above, that comprise the protein or immunogenic fragments thereof according to the invention.

All vaccines described above contribute to active vaccination, i.e. they trigger the host's defense system.

Alternatively, antibodies can be raised in e.g. rabbits or can be obtained from antibody-producing cell lines as described below. Such antibodies can then be administered to the pig. This method of vaccination, passive vaccination, is the vaccination of choice when an animal is already infected, and there is no time to allow the natural immune response to be triggered. It is also the preferred method for vaccinating animals that are prone to sudden high infection pressure. The administered antibodies against the protein according to the invention or immunogenic fragments thereof can in these cases bind directly to Brachyspira hyodysenteriae. This has the advantage that it decreases or stops Brachyspira hyodysenteriae multiplication.

Therefore, one other form of this embodiment of the invention relates to a vaccine for combating Brachyspira hyodysenteriae infection that comprises antibodies against a Brachyspira hyodysenteriae protein according to the invention or an immunogenic fragment of that protein, and a pharmaceutically acceptable carrier.

Still another embodiment of this invention relates to antibodies against a Brachyspira hyodysenteriae protein according to the invention or an immunogenic fragment of that protein.

Methods for large-scale production of antibodies according to the invention are also known in the art. Such methods rely on the cloning of (fragments of) the genetic information encoding the protein according to the invention in a filamentous phage for phage display. Such techniques are described i.a. at the “Antibody Engineering Page” under “filamentous phage display” at http://aximt1.imt.uni-marburg.de/˜rek/aepphage.html., and in review papers by Cortese, R. et al., (1994) in Trends Biotechn. 12: 262-267., by Clackson, T. & Wells, J. A. (1994) in Trends Biotechn. 12: 173-183, by Marks, J. D. et al., (1992) in J. Biol. Chem. 267:16007-16010, by Winter, G. et al., (1994) in Annu. Rev. Immunol. 12: 433-455, and by Little, M. et al., (1994) Biotechn. Adv. 12: 539-555. The phages are subsequently used to screen camelid expression libraries expressing camelid heavy chain antibodies. (Muyldermans, S. and Lauwereys, M., Journ. Molec. Recogn. 12:131-140 (1999) and Ghahroudi, M. A. et al., FEBS Letters 414: 512-526 (1997)). Cells from the library that express the desired antibodies can be replicated and subsequently be used for large scale expression of antibodies.

Still another embodiment relates to a method for the preparation of a vaccine according to the invention that comprises the admixing of antibodies according to the invention and a pharmaceutically acceptable carrier.

An alternative and efficient way of vaccination is direct vaccination with DNA encoding the relevant antigen. Direct vaccination with DNA encoding proteins has been successful for many different proteins. (As reviewed in e.g. Donnelly et al., The Immunologist 2: 20-26 (1993)). This way of vaccination is also attractive for the vaccination of pigs against Brachyspira hyodysenteriae infection. Therefore, still other forms of this embodiment of the invention relate to vaccines comprising nucleic acid sequences encoding a protein according to the invention or immunogenic fragments thereof, comprising DNA fragments that comprise such nucleic acid sequences or comprising recombinant DNA molecules according to the invention, and a pharmaceutically acceptable carrier.

Examples of DNA plasmids that are suitable for use in a DNA vaccine according to the invention are conventional cloning or expression plasmids for bacterial, eukaryotic and yeast host cells, many of said plasmids being commercially available. Well-known examples of such plasmids are pBR322 and pcDNA3 (Invitrogen). The DNA fragments or recombinant DNA molecules according to the invention should be able to induce protein expression of the nucleotide sequences. The DNA fragments or recombinant DNA molecules may comprise one or more nucleotide sequences according to the invention. In addition, the DNA fragments or recombinant DNA molecules may comprise other nucleotide sequences such as the immune-stimulating oligonucleotides having unmethylated CpG di-nucleotides, or nucleotide sequences that code for other antigenic proteins or adjuvating cytokines.

The nucleotide sequence according to the present invention or the DNA plasmid comprising a nucleotide sequence according to the present invention, preferably operably linked to a transcriptional regulatory sequence, to be used in the vaccine according to the invention can be naked or can be packaged in a delivery system. Suitable delivery systems are lipid vesicles, iscoms, dendromers, niosomes, polysaccharide matrices and the like, (see further below) all well-known in the art. Also very suitable as delivery system are attenuated live bacteria such as Salmonella species, and attenuated live viruses such as Herpesvirus vectors, as mentioned above.

Still other forms of this embodiment relate to vaccines comprising recombinant DNA molecules according to the invention.

DNA vaccines can e.g. easily be administered through intradermal application such as by using a needle-less injector. This way of administration delivers the DNA directly into the cells of the animal to be vaccinated. Amounts of DNA in the range between 10 pg and 1000 μg provide good results. Preferably, amounts in the microgram range between 1 and 100 μg are used.

In a further embodiment, the vaccine according to the present invention additionally comprises one or more antigens derived from pig pathogenic organisms and viruses, antibodies against those antigens or genetic information encoding such antigens.

Of course, such antigens can be e.g. other Brachyspira hyodysenteriae antigens. It can also be an antigen selected from another other pig pathogenic organism or virus. Such organisms and viruses are preferably selected from the group of Pseudorabies virus, Porcine influenza virus, Porcine parvo virus, Transmissible gastro-enteritis virus, Rotavirus, Escherichia coli Erysipelo rhusiopathiae, Bordetella bronchiseptica, Salmonella cholerasuis, Haemophilus parasuis, Pasteurella multocida, Streptococcus suis, Mycoplasma hyopneumoniae and Actinobacillus pleuropneumoniae.

Vaccines based upon the 61 kD and/or 20 kD Brachyspira hyodysenteriae lipoprotein are also very suitable as marker vaccines. A marker vaccine is a vaccine that allows to discriminate between vaccinated and field-infected pigs e.g. on the basis of a characteristic antibody panel, different from the antibody panel induced by wild type infection. A different antibody panel is induced e.g. when an immunogenic protein present on a wild type bacterium is not present in a vaccine: the host will then not make antibodies against that protein after vaccination. Thus, a vaccine based upon the 61 kD and/or 20 kD Brachyspira hyodysenteriae lipoprotein according to the invention would only induce antibodies against the 61 kD and/or 20 kD lipoprotein, whereas a vaccine based upon a live wild-type, live attenuated or inactivated whole Brachyspira hyodysenteriae would induce antibodies against all or most of the bacterial proteins.

A simple ELISA test, having wells comprising e.g. the purified recombinant nucleoprotein and wells comprising only purified 61 kD and/or 20 kD Brachyspira hyodysenteriae lipoprotein suffices to test serum from pigs and to tell if the pigs are either vaccinated with the 61 kD and/or 20 kD lipoprotein vaccine or suffered from Brachyspiral field infection.

All vaccines according to the present invention comprise a pharmaceutically acceptable carrier. A pharmaceutically acceptable carrier can be e.g. sterile water or a sterile physiological salt solution. In a more complex form the carrier can e.g. be a buffer.

Methods for the preparation of a vaccine comprise the admixing of a protein or an immunogenic fragment thereof, according to the invention and/or antibodies against that protein or an immunogenic fragment thereof, and/or a nucleic acid sequence and/or a DNA fragment, a recombinant DNA molecule, a live recombinant carrier or host cell according to the invention, and a pharmaceutically acceptable carrier.

Vaccines according to the present invention may in a preferred presentation also contain an immunostimulatory substance, a so-called adjuvant. Adjuvants in general comprise substances that boost the immune response of the host in a non-specific manner. A number of different adjuvants are known in the art. Examples of adjuvants frequently used in pig vaccines are muramyldipeptides, lipopolysaccharides, several glucans and glycans and Carbopol® (a homopolymer).

The vaccine may also comprise a so-called “vehicle”. A vehicle is a compound to which the protein adheres, without being covalently bound to it. Such vehicles are i.a. bio-microcapsules, micro-alginates, liposomes and macrosols, all known in the art.

A special form of such a vehicle, in which the antigen is partially embedded in the vehicle, is the so-called ISCOM (EP 109.942, EP 180.564, EP 242.380)

In addition, the vaccine may comprise one or more suitable surface-active compounds or emulsifiers, e.g. Span or Tween.

Often, the vaccine is mixed with stabilisers, e.g. to protect degradation-prone proteins from being degraded, to enhance the shelf-life of the vaccine, or to improve freeze-drying efficiency. Useful stabilisers are i.a. SPGA (Bovarnik et al; J. Bacteriology 59: 509 (1950)), carbohydrates e.g. sorbitol, mannitol, trehalose, starch, sucrose, dextran or glucose, proteins such as albumin or casein or degradation products thereof, and buffers, such as alkali metal phosphates.

In addition, the vaccine may be suspended in a physiologically acceptable diluent.

It goes without saying, that other ways of adjuvating, adding vehicle compounds or diluents, emulsifying or stabilising a protein are also embodied in the present invention.

Vaccines according to the invention that are based upon the protein according to the invention or immunogenic fragments thereof can very suitably be administered in amounts ranging between 1 and 100 micrograms of protein per animal, although smaller doses can in principle be used. A dose exceeding 100 micrograms will, although immunologically very suitable, be less attractive for commercial reasons.

Vaccines based upon live attenuated recombinant carriers, such as the LRC-viruses and bacteria described above can be administered in much lower doses, because they multiply themselves during the infection. Therefore, very suitable amounts would range between 10³ and 10⁹ CFU/PFU for respectively bacteria and viruses.

Vaccines according to the invention can be administered e.g. intradermally, subcutaneously, intramuscularly, intraperitoneally, intravenously, or at mucosal surfaces such as orally or intranasally.

For efficient protection against disease, a quick and correct diagnosis of Brachyspiral infection is important.

Therefore it is another objective of this invention to provide diagnostic tools suitable for the detection of Brachyspiral infection.

The nucleic acid sequences, the proteins and the antibodies according to the invention are also suitable for use in diagnostics.

Therefore, another embodiment of the invention relates to nucleic acid sequences, proteins and antibodies according to the invention for use in diagnostics.

The nucleic acid sequences or fragments thereof according to the invention can be used to detect the presence of Brachyspira in pigs. A sample taken from pigs infected with Brachyspira will comprise nucleic acid material derived from said bacterium, including nucleic acid sequences encoding for the protein according to the invention. These nucleic acid sequences will hybridize with a nucleic acid sequence according to the invention. Suitable methods for the detection of nucleic acid sequences that are reactive with the nucleic acid sequences of the present invention include hybridization techniques including but not limited to PCR techniques and NASBA techniques. Thus the nucleic acid sequences according to the invention, in particular the sequences depicted in SEQ ID NO 1 and/or SEQ ID NO 3 can be used to prepare probes and primers for use in PCR and or NASBA techniques.

A diagnostic test kit for the detection of Brachyspira hyodysenteriae may e.g. comprise tools to enable the reaction of bacterial nucleic acid isolated from the pigs to be tested with these tools. Such tools are e.g. specific probes or (PCR-) primers, also referred to as primer fragments, based upon the nucleic acid sequences according to the invention. If genetic material of B. hyodysenteriae is present in the animal, this will e.g. specifically bind to specific PCR-primers and, e.g. after cDNA synthesis, will subsequently become amplified in PCR-reaction. The PCR-reaction product can then easily be detected in DNA gel electrophoresis.

Standard PCR-textbooks give methods for determining the length of the primers for selective PCR-reactions with Brachyspira hyodysenteriae DNA. Primer fragments with a nucleotide sequence of at least 12 nucleotides are frequently used, but primers of more than 15, more preferably 18 nucleotides are somewhat more selective. Especially primers with a length of at least 20, preferably at least 30 nucleotides are very generally applicable. PCR-techniques are extensively described in Dieffenbach & Dreksler; PCR primers, a laboratory manual. ISBN 0-87969-447-5 (1995).

Nucleic acid sequences according to the invention or primers of those nucleic acid sequences having a length of at least 12, preferably 15, more preferably 18, even more preferably 20, 22, 25, 30, 35 or 40 nucleotides in that order of preference, wherein the nucleic acid sequences or parts thereof have at least 70% homology with the nucleic acid sequence as depicted in SEQ ID NO: 1 and/or SEQ ID NO 3 are therefore also part of the invention. Primers are understood to have a length of at least 12 nucleotides and a homology of at least 70%, more preferably 80%, 85%, 90%, 95%, 98%, 99% or even 100%, in that order of preference, with the nucleic acid sequence as depicted in SEQ ID NO: 1 or SEQ ID NO 3. Such nucleic acid sequences can be used as primer fragments in PCR-reactions in order to enhance the amount of DNA that they encode or in hybridization reactions. This allows the quick amplification or detection on blots of specific nucleotide sequences for use as a diagnostic tool for e.g. the detection of Brachyspira hyodysenteriae as indicated above.

Another test on genetic material is based upon growth of bacterial material obtained from e.g. a swab, followed by classical DNA purification followed by classical hybridization with radioactively or color-labeled primer fragments. Colour-labelled and radioactively labeled fragments are generally called detection means. Both PCR-reactions and hybridization reactions are well-known in the art and are i.a. described in Maniatis/Sambrook (Sambrook, J. et al. Molecular cloning: a laboratory manual. ISBN 0-87969-309-6).

Thus, one embodiment of the invention relates to a diagnostic test kit for the detection of Brachyspira hyodysenteriae nucleic acid sequences. Such a test comprises a nucleic acid sequence according to the invention or a primer fragment thereof.

A diagnostic test kit based upon the detection of antigenic material of the specific Brachyspira hyodysenteriae 61 kD and/or 20 kD lipoprotein and therefore suitable for the detection of Brachyspira hyodysenteriae infection may i.a. comprise a standard ELISA test. In one example of such a test the walls of the wells of an ELISA plate are coated with antibodies directed against the 61 kD and/or 20 kD lipoprotein. After incubation with the material to be tested, labeled anti-Brachyspira hyodysenteriae antibodies are added to the wells. A color reaction then reveals the presence of antigenic material from Brachyspira hyodysenteriae.

Therefore, still another embodiment of the present invention relates to diagnostic test kits for the detection of antigenic material of Brachyspira hyodysenteriae. Such test kits comprise antibodies against a 61 kD and/or 20 kD lipoprotein or a fragment thereof according to the invention.

A diagnostic test kit based upon the detection in serum of antibodies against the 61 kD and/or 20 kD lipoprotein of Brachyspira hyodysenteriae and therefore suitable for the detection of Brachyspira hyodysenteriae infection may i.a. comprise a standard ELISA test. In such a test the walls of the wells of an ELISA plate can e.g. be coated with the 61 kD or 20 kD lipoprotein. After incubation with the material to be tested, labeled anti-61 kD or 20 kD antibodies are added to the wells. A lack of color reaction then reveals the presence of antibodies against Brachyspira hyodysenteriae.

Therefore, still another embodiment of the present invention relates to diagnostic test kits for the detection of antibodies against Brachyspira hyodysenteriae. Such test kits comprise the 61 kD and/or 20 kD Brachyspira hyodysenteriae lipoprotein or a fragment thereof according to the invention.

The design of the immunoassay may vary. For example, the immunoassay may be based upon competition or direct reaction. Furthermore, protocols may use solid supports or may use cellular material. The detection of the antibody-antigen complex may involve the use of labeled antibodies; the labels may be, for example, enzymes, fluorescent-, chemoluminescent-, radio-active- or dye molecules.

Suitable methods for the detection of antibodies reactive with a protein according to the present invention in the sample include the enzyme-linked immunosorbent assay (ELISA), immunofluorescense test (IFT) and Western blot analysis.

The proteins or immunogenic fragments thereof according to the invention e.g. expressed as indicated above can be used to produce antibodies, which may be polyclonal, monospecific or monoclonal (or derivatives thereof). If polyclonal antibodies are desired, techniques for producing and processing polyclonal sera are well-known in the art (e.g. Mayer and Walter, eds. Immunochemical Methods in Cell and Molecular Biology, Academic Press, London, 1987).

Monoclonal antibodies, reactive against the protein according to the invention or an immunogenic fragment thereof according to the present invention, can be prepared by immunizing inbred mice by techniques also known in the art (Kohler and Milstein, Nature, 256, 495-497,1975).

### EXAMPLES

### Example 1

Bacterial strains and media. B. hyodysenteriae B204^{T} was used in this study. Brachyspira were grown anaerobically at 37° C. for 48 h on trypticase soy agar containing 5% defibrinated horse blood supplemented with 0.1% yeast extract. Broth cultures of B. hyodysenteriae were prepared as described by Wannemuehler et al. (Wannemuehler, M. J., R. D. Hubbard, and J. M. Greer. 1988. Characterization of the major outer membrane antigens of Treponema hyodysenteriae. Infect. Immun. 56:3032-3039). E. coli DH5α was used for cloning and construction of a gene library. E. coli KSS330r⁻ [F⁻Δ(ara-leu)7697 galE galK ΔlacX74 rpsL (Str^{r}) degP4::Tn5 Ipp5508] (Strauch, K. L., and J. Beckwith. 1988, Proc. Natl. Acad. Sci. USA 85:1576-1580). was used to check plasmid inserts for the blue halo phenotype. E. coli strains were cultured in Luria-Bertani (LB) broth or on 1.5% LB agar at 37° C. overnight.

DNA manipulations. Chromosomal DNA from B. hyodysenteriae was prepared using the cetyltrimethylammonium bromide precipitation method (Ausubel, F. A., R. Brent, R. E. Kingston, D. D. Moore, J. G. Seidman, J. A. Smith, and K. Struhl. 1991, Current protocols in molecular biology. Greene Publishing and Wiley Interscience, New York), while plasmid DNA was isolated as described by Birnboim and Doly (Birnboim, H. C., and J. Doly. 1979, Nucleic Acids Res. 7:1513-1523).

Standard methods in molecular biology were performed essentially as described by Sambrook et al. (Sambrook, J., E. F. Fritsch, and T. Maniatis. 1989, Molecular cloning: a laboratory manual. Cold Spring Harbour Laboratory Press, Cold Spring Harbour, N.Y.). Nucleotide sequencing was performed using the BigDye DyeDeoxy Terminator cycle sequencing kit (The Perkin-Elmer Corp., Norwalk, Conn.) and an Applied Biosystems Inc. 373A automated sequencer.

Blue halo assay. Genomic libraries of B. hyodysenteriae were constructed using the signal peptide-deficient alkaline phosphatase vector pMG and analyzed as described previously (Blanco, D. R., M. Giladi, C. I. Champion, D. A. Haake, G. K. Chikami, J. N. Miller, and M. A. Lovett. 1991, Mol. Microbiol. 5:2405-2415), (Giladi, M., Champion, C. I., Haake, D. A., Blanco, D. R., Miller, J. F. & Lovett, M. A. (1993). Use of the “blue halo” assay in the identification of genes encoding exported proteins with cleavable signal peptides: Cloning of a Borrelia burgdorferi gene with a signal peptide. Journal of Bacteriology 175, 4129-4136).

Construction of Genomic Library in pBluescript II SK

Chromosomal DNA from B. hyodysenteriae strain B204 was isolated by standard methods and digested with HindIII according to the manufacturers directions. The restriction enzyme was removed by phenol extraction and ethanol precipitation. 5 μg of the digested chromosomal DNA was mixed with 0.1 μg of HindIII digested pBluescript II SK (Stratagene Co.). To this mixture 5 units of T4 DNA ligase (Gibco-BRL) was added and incubated for 18 hours at 14° C. E. coli strain DH5α was made competent (Hanahan) and transformed with 10 μl of the ligation mix and after recovery for 1 hour at 37° C. in Luria Bertani broth the mixture was plated on Luria Bertani plates supplemented with 100 μg/ml of ampicillin. A total of >4000 colonies was obtained. A total of 4000 colonies from the obtained HindIII library were plated on LB plates with 100 μg/ml of ampicillin and grown for 18 hours at 37° C. Colonies were lifted onto nitrocellulose filters, fixed in chloroform vapor and then lysed with SDS using standard methods (Sambrook et al.). The obtained filters were incubated with serum obtained from a pig with B. hyodysenteriae infection. The colony blots were developed with rabbit-anti pig alkaline phosphatase conjugated secondary antibodies. Colonies reacting with the convalescent serum were purified and plasmid DNA was extracted from cultures grown in LB medium with 100 μg/ml of ampicillin.

Clone HBA3 was shown to contain an 1.8 kb HindIII insert from which the sequence was determined. An ORF of 1614 bp was identified (SEQ ID NO 1) which encodes a lipoprotein designated BlpB (SEQ ID NO 2). The lipoprotein has a calculated molecular mass of 60.8 kD.

Construction and Screening of Blue Halo Libraries

Genomic libraries of B. hyodysenteriae (B204) were constructed using the signal peptide deficient alkaline phosphatase vector, pMG (Giladi et al, 1993). A mixture of pMG 1c, 2.7 and 3.29 vectors were digested with SmaI to allow cloning in all three reading frames. Aliquots of genomic DNA were completely digested with AluI and RsaI and ligated to alkaline phosphatase-treated vectors. Electrocompetent E. coli DH5α cells were transformed with the ligation mix, incubated with shaking for 1 hour at 37° C. to allow expression, and then plated onto LB agar containing ampicillin, 1 mM IPTG and 40 μg/ml XP (5-Bromo-4-chloro-3-indoyl phosphate) to identify blue colonies, containing inserts partially encoding membrane and exported proteins. Approximately 1700 recombinants were obtained, of which 23 exhibited the blue phenotype. Transformation of the plasmid DNA from the 23 blue colonies into E. coli KSS330r⁻ and plating on to 1 mM IPTG and 200 μg/ml XP media resulted in 5 colonies expressing the blue halo phenotype. Plasmid DNA was isolated from colonies expressing the blue halo phenotype and used as template in PCRs with vector primers which flank the polylinker, in order to determine the size of the plasmid inserts. Sequence analysis with one of the vector primers on the obtained plasmids resulted in the 5′end of the blpC gene. The remainder of the gene sequence was obtained by SSP-PCR (Shyamala & Ames, Gene 84, 1-8, 1989). An ORF of 537 bp was identified (SEQ ID NO 3) which encodes a lipoprotein designated BlpC (SEQ ID NO 4). The lipoprotein has a calculated molecular mass of 20 kD.

### Example 2

Expression of BlpB and BlpC in E. coli

PCR was used to amplify the genes encoding the mature length proteins BlpB and BlpC using primers designed to engineer unique restriction endonuclease sites into the final product. The 5′ primers incorporate either an NdeI site or a BamH1 site. The 3′ primers incorporate a BamHI site or an NdeI site. An aliquot of the PCR products were visualized on an agarose gel and the remaining portions were digested with BamHI (Roche) and/or NdeI (Roche) according to manufactures instructions. Restriction endonuclease were inactivated by phenol extraction and the digested PCR products were purified by ethanol precipitation. The pET-15b vector was digested and purified as per the PCR products and treated with alkaline phosphatase. 150 μg of the digested PCR products were ligated into 80 ng of digested pET15-b using T4 DNA ligase (Roche) according to manufactures instructions. The ligations were transformed into E. coli DH5α. Clones containing single inserts were identified using colony PCR with primers designed to flank the multicloning site of the vector: 5′-TAA-TAC-GAC-TCA-CTA-TAG-G-3′ and 5′-GGA-MC-AGC-TAT-GAC-CAT-G-3′. For each ligation the insert of one of the clones was checked by double stranded DNA sequencing. Plasmid DNA was isolated from the positive clone and transformed into E. coli strain BL21 (DE3) pLysS for expression. To check expression of recombinant proteins whole cell lysates of the induced cultures of the expression clones were compared with the expression strain harboring a pET15b plasmid without an insert. Whole cell lysates were separated by SDS-PAGE (as described previously) and stained with coomasie brillant blue (CBB). Bands corresponding to the predicted molecular weights of all the recombinant proteins were observed except for BlpB. By transformation of the blpB.pET15b construct into CodonPlus RIL (Stratagene) strain a band corresponding to its molecular weight was also observed.

Reaction of the Recombinant Blps with Convalescent Serum

To check whether the recombinant proteins were recognized by convalescent sera whole cell lysates of the induced cultures of the clones expressing BlpB and BlpC were separated by SDS-PAGE alongside the expression strain harboring a pET15b plasmid without an insert. Proteins were then transferred onto Immobilon-P membranes (Millipore) with a Trans-Blot® Electrophoretic transfer Cell (Bio-Rad) for 1 hour at 100 V according to the manufactures instructions. After transfer, the membranes were blocked by soaking in 5% skim milk buffer [5% (w/v) skim milk powder in TBS-Tween 20 (0.15 M NaCl, 0.05 M Tris-HCl pH 7.4, 0.05% Tween 20)] for 1 hr. Serum obtained from a pig with B. hyodysenteriae infection was diluted 1/100 in skim milk buffer, and incubated overnight. Three 5-minute washes in TBS-Tween were performed before incubating the membranes with the secondary antibody [1/400 dilution of horse-radish peroxidase (HRPO)—conjugated rabbit-anti pig IgG (Sigma)]. The secondary antibody was diluted in skim milk buffer and incubated with the membranes for 2 hours at 37° C. with shaking, followed by three 5-minute washes as above, with the addition of a final 5 minute wash in TBS. The membranes were developed using HRPO substrate [4-chloro-1-napthol (Merck), 100% methanol, 30% (w/v) H₂O₂ TBS].

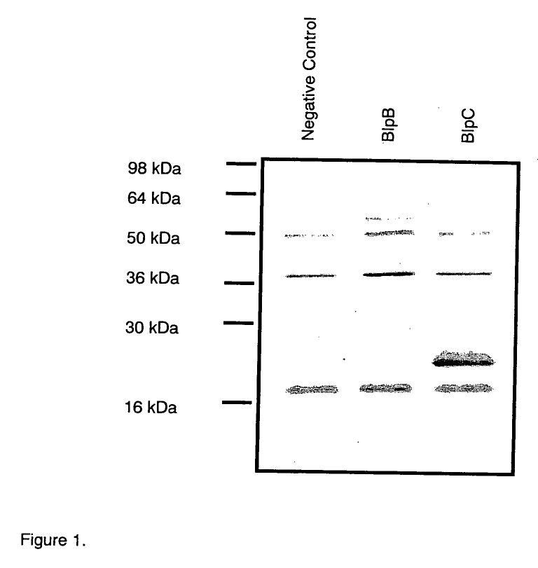

Bands corresponding to the predicted molecular weights of BlpB and BlpC were observed for each of their respective expression clones (FIG. 1).

Production of Antisera Against the Recombinant Blps

Cultures of strains expressing recombinant BlpC were grown to an optical density of 0.6 at 600 nm and induced for 4 hrs with 10 mM isopropylthio-β-D-galactoside (IPTG; Sigma). Cultures were lysed using a French pressure cell and recombinant proteins were purified using Talon resin (Clontech) by immobilised metal affinity chromatography (IMAC) according to the manufactures instructions. The column eluates were pooled and dialysed overnight against phosphate buffered saline and concentrated to a 0.5 mL volume using Centicon-10 (Millipore) concentrators. The concentration of sample was determined by Bradford assay. Freund's incomplete adjuvant was combined in equal volumes with 100 μg of each of the recombinant proteins. Two New Zealand White rabbits were injected subcutaneously with 50 μg of purified recombinant protein. After 4 weeks the rabbits were injected with another 50 μg of purified recombinant protein intradermally. After an additional week the rabbits were subjected to a terminal bleed by cardiac puncture. The rabbit antisera were shown to react with recombinant protein in Western blotting experiments at dilutions at 1/2000.

Legend to the Figures.

FIG. 1: Western blots of hole cell lysates from induced blpB and blpC expression clones reactive with pig serum (1/100 dilution).

1-4. (canceled)

5. A DNA fragment comprising a nucleic acid sequence according to claim 25.

6. A recombinant DNA molecule comprising a nucleic acid sequence according to claim 25, under the control of a functionally linked promoter.

7. A live recombinant carrier comprising a nucleic acid sequence according to claim 25.

8. A host cell comprising a nucleic acid sequence according to claim 25.

9-18. (canceled)

19. A vaccine for combating Brachyspira hyodysenteriae infection comprising antibodies against a lipoprotein according to claim 29, and a pharmaceutically acceptable carrier.

20. The vaccine according to claim 19, comprising an adjuvant.

21. The vaccine according to claim 19, comprising an additional antigen derived from another swine pathogen, an antibody against such an antigen or genetic information encoding said antigen.

22. The vaccine according to claim 21, wherein said pathogen is selected from the group consisting of Pseudorabies virus, Porcine influenza virus, Porcine parvo virus, Transmissible gastro-enteritis virus, Rotavirus, Escherichia coli, Erysipelo rhusiopathiae, Bordetella bronchiseptica, Salmonella cholerasuis, Haemophilus parasuis, Pasteurella multocida, Streptococcus suis, Mycoplasma hyopneumoniae and Actinobacillus pleuropneumoniae.

23. (canceled)

24. A diagnostic kit for detecting Brachyspira hyodysenteriae or antibodies thereto, comprising a nucleic acid sequence according to claim 25, or a lipoprotein or antigenic fragment thereof encoded by said sequence, or antibodies that are reactive with said lipoprotein.

25. A nucleic acid sequence encoding an immunogenic 61 kD Brachyspira hyodysenteriae lipoprotein, as measured by SDS-PAGE.

26. The nucleic acid sequence of claim 25, wherein the 61 kD Brachyspira hyodysenteriae lipoprotein has the amino acid sequence of SEQ ID NO:2.

27. A nucleic acid sequence encoding an immunogenic 20 kD Brachyspira hyodysenteriae lipoprotein, as measured by SDS-PAGE.

28. The nucleic acid sequence of claim 27, wherein the 20 kD Brachyspira hyodysenteriae lipoprotein has the amino acid sequence of SEQ ID NO:4.

29. An isolated immunogenic Brachyspira hyodysenteriae lipoprotein of 61 kD, as measured by SDS-PAGE.

30. The isolated immunogenic Brachyspira hyodysenteriae lipoprotein of claim 29 having the amino acid sequence of SEQ ID NO: 2.

31. An isolated immunogenic Brachyspira hyodysenteriae lipoprotein of 20 kD, as measured by SDS-PAGE.

32. The isolated immunogenic Brachyspira hyodysenteriae lipoprotein of claim 31 having the amino acid sequence of SEQ ID NO:4.

33. An immunogenic composition comprising an effective amount of the Brachyspira hyodysenteriae lipoprotein of claim 29 and a pharmaceutically acceptable carrier.

34. An immunogenic composition comprising an effective amount of the Brachyspira hyodysenteriae lipoprotein of claim 31 and a pharmaceutically acceptable carrier.

35. An immunogenic composition comprising an immunogenically effective amount of a nucleic acid sequence selected from the group consisting of a DNA fragment, a recombinant DNA molecule and a live recombinant carrier, or host cell comprising said nucleic acid sequence, wherein said nucleic acid sequence is the nucleic acid sequence of claim 25.

36. An immunogenic composition comprising an immunogenically effective amount of a nucleic acid sequence selected from the group consisting of a DNA fragment, a recombinant DNA molecule and a live recombinant carrier, or a host cell comprising said nucleic sequence, wherein said nucleic acid sequence is the nucleic acid sequence of claim 27.

37. A DNA fragment comprising a nucleic acid sequence according to claim 27.

38. A recombinant DNA molecule comprising a nucleic acid sequence according to claim 27, under the control of a functionally linked promoter.

39. A live recombinant carrier comprising a nucleic acid sequence according to claim 27.

40. A host cell comprising a nucleic acid sequence according to claim 27.

41. A diagnostic kit for detecting Brachyspira hyodysenteriae or antibodies thereto, comprising a nucleic acid sequence according to claim 27, or a lipoprotein or antigenic fragment thereof encoded by said sequence, or antibodies that are reactive with said lipoprotein.

42. A vaccine for combating Brachyspira hyodysenteriae infection, comprising antibodies against a lipoprotein according to claim 31 and a pharmaceutically acceptable carrier.

43. The vaccine according to claim 42, comprising an adjuvant.

44. The vaccine according to claim 42, comprising an additional antigen derived from another swine pathogen, an antibody against such an antigen or genetic information encoding said antigen.

45. The vaccine according to claim 44, wherein said pathogen is selected from the group constituting of Pseudorabies virus, Porcine influenza virus, Porcine parvo virus, Transmissible gastro-enteritis virus, Rotavirus, Escherichia coli, Erysipelo rhusiopathiae, Bordetella bronchiseptica, Salmonella cholerasuis, Haemophilus parasuis, Pasteurella multocida, Streptococcus suis, Mycoplasma hyopneumoniae and Actinobacillus pleuropneumoniae.


SEQUENCE LISTING (4 sequences)

SEQ ID NO: 1 - DNA, 1614 bp, Brachyspira hyodysenteria
  CDS (1)..(1611)
atgaaaaaactatattcattatttatatttatagctgtaattttatttatttattcatgcggaagttattttaatcctaaatactatttttttaaaagtaaagtagagaataatggaagttctggaagctcaggcggtaattcaggaatatatatacagcctggtgaagatgaagatccttttacagcacctaaatatgcatctgagtggtggaatgatcctaataatggtggatttgatgcttcggatatagataaatggtttttgaaagttgaatttttagcaaatgattatccaagttacagatttttaacaaaaagcggaagaaaagcaggacatgtttgggtagtttctaatgaacaatctcaggcatatttagatcaaggagaagtatgcaatactacagcagttaccggggtatctataaaaccagtagcaaatgtaacaggagtaacatatacaaaatataagggattgaatgcaagatttttcactcatgacggagattatcataatgtatatcctggaaaagataagataagcagattctatttttattattttacaggtactccggaaatggctaaatttttagaaaattgcttaatagctgtagacacatactcaaaactattattttactatggaagacctcaaagcgattatccaaatcctccaagctggcagaagcctagtaatttggttgataaatactctcctacaggttattggatatctatagatgaaggtataaatgataaggggcagaattatcctttttatgaatatgatcctgtaggatatgtaaaaagcgatggtacagttgttatatttgattggtttgcaaacagattaagaggaaatcataataatgatcctataaaaagtgatcctaaaggagctattgttcctaatcctaatactaatccttctgcttcaactacaggacgttctccttatgcattctattcgcctttagcacaaaaagataaaactaaaataactataagtactacaaaattaataaattatacagttttcagttataaatacagtatacaaatatttccgcctagtatgaaagaagaaaaacttccttatgcttatattgcatatgcttcctatggtgcagcttatcaaaatgaaagcagcaaatctgtagagatgatatctgatataaataaaggagagcattacggaagtattacaagaataagcactgtacctaaaatagataaagacggaggagagttggtaaaagaaggatctaagtcttttgaattatatggtattgatactaaagatacatttatagaattaagtttaaaactaattaaaaatgatgaaaatacagaatttgttgatcaaggaacagcaggtactggtcctttagtatactttgataaaacagatcctatacttgtattaaaatatgataaatcttctgacagcttcaaatacagcagtgtaaaaggaaacaagcaaatagaagttgacagcaatttatcaattaaaagaggagagaataaagagtttacagttaaatttaaagatccaaataatggaaatgagtttggggttgtatttaaaatagattttgaaaaaatatcatga

SEQ ID NO: 2 - protein, 537 aa, Brachyspira hyodysenteria
MKKLYSLFIFIAVILFIYSCGSYFNPKYYFFKSKVENNGSSGSSGGNSGIYIQPGEDEDPFTAPKYASEWWNDPNNGGFDASDIDKWFLKVEFLANDYPSYRFLTKSGRKAGHVWVVSNEQSQAYLDQGEVCNTTAVTGVSIKPVANVTGVTYTKYKGLNARFFTHDGDYHNVYPGKDKISRFYFYYFTGTPEMAKFLENCLIAVDTYSKLLFYYGRPQSDYPNPPSWQKPSNLVDKYSPTGYWISIDEGINDKGQNYPFYEYDPVGYVKSDGTVVIFDWFANRLRGNHNNDPIKSDPKGAIVPNPNTNPSASTTGRSPYAFYSPLAQKDKTKITISTTKLINYTVFSYKYSIQIFPPSMKEEKLPYAYIAYASYGAAYQNESSKSVEMISDINKGEHYGSITRISTVPKIDKDGGELVKEGSKSFELYGIDTKDTFIELSLKLIKNDENTEFVDQGTAGTGPLVYFDKTDPILVLKYDKSSDSFKYSSVKGNKQIEVDSNLSIKRGENKEFTVKFKDPNNGNEFGVVFKIDFEKIS

SEQ ID NO: 3 - DNA, 540 bp, Brachyspira hyodysenteria
  CDS (1)..(537)
atgtttaagaaactttttattgttgtgggtttgatgagtgtgcttgctggatgtgaaactatgcagcctaaaaataatgatacaattgtaaaaaatgataattcatctaatgaagataaaaaagaagaaactataactagagaagatacaccaaaaatgaaagttacagtttatggagcagataaagaaattcaagctgtggaaataaatgataaaacttattatgtaataggcggaaaagatgttgagaatatgacagaagctgatataaaaaaatcatctttagtagcacctttaaaagttacagaagaaactgttaatggtacaagaggtatagttgttacatattatgatgtaaaagtattcttgggtaaaagaacaggaacaggaactatcgtaggaatattcgagcctcagaaaaatgattggactacaggaaatgatttggatagaagtttatctattcaaataaaattatctagaaatatagcaggtcctatagatataaaaagaggaagcatatctttagcatttaattaa

SEQ ID NO: 4 - protein, 179 aa, Brachyspira hyodysenteria
MFKKLFIVVGLMSVLAGCETMQPKNNDTIVKNDNSSNEDKKEETITREDTPKMKVTVYGADKEIQAVEINDKTYYVIGGKDVENMTEADIKKSSLVAPLKVTEETVNGTRGIVVTYYDVKVFLGKRTGTGTIVGIFEPQKNDWTTGNDLDRSLSIQIKLSRNIAGPIDIKRGSISLAFN